(19) United States
(12) Patent Application Publication
(10) Pub. No.: US 2023/0038479 A1
(43) Pub. Date: Feb. 9, 2023

(54) GENE THERAPY

(71) Applicant: University of Sheffield, Sheffield (GB)

(72) Inventors: Mimoun Azzouz, Sheffield (GB); Christopher Webster, Sheffield (GB)

(73) Assignee: University of Sheffield, Sheffield (GB)

(21) Appl. No.: 17/791,169

(22) PCT Filed: Feb. 2, 2021
(86) PCT No.: PCT/EP2021/052378

§ 371 Date: Jul. 6, 2022

(30) Foreign Application Priority Data
Feb. 12, 2020 (GB) 2001930.3

(51) Int. Cl. C12N 9/14; C12N 15/86; A61P 25/28

(52) U.S. Cl. CPC C12N 9/14; C12Y 306/04012; C12N 15/86; A61P 25/28; C12N 2750/14143

(57) ABSTRACT

The present disclosure relates to transcription cassettes comprising nucleic acids encoding RuvBL1 and/or RuvBL2 and the use of said vectors in gene therapy for the treatment of neurodegenerative diseases that result from expression of polymorphic repeat expansions of the GGGGCC (SEQ ID NO: 5) hexanucleotide-repeat sequence in the first intron of the C9ORF72 gene; pharmaceutical compositions comprising said vectors and including uses and methods to treat neurodegenerative diseases.

### CROSS REFERENCE TO RELATED APPLICATIONS

This is the U.S. National Stage of International Application No. PCT/EP2021/052378, filed Feb. 2, 2021, which was published in English under PCT Article 21(2), which in turn claims the benefit of Great Britain Application No. GB 2001930.3, filed Feb. 12, 2020.

### FIELD OF THE DISCLOSURE

The present disclosure relates to a transcription cassette comprising nucleic acids encoding RuvBL1 and/or RuvBL2 and the use of said vectors in gene therapy for the treatment of neurodegenerative diseases that result from expression of polymorphic repeat expansions of the GGGGCC (SEQ ID NO: 5) hexanucleotide-repeat sequence in the first intron of the C9ORF72 gene; pharmaceutical compositions comprising said vectors and including uses and methods to treat neurodegenerative diseases.

### BACKGROUND THE DISCLOSURE

Amyotrophic lateral sclerosis (ALS) and frontotemporal dementia (FTD) are adult onset neurodegenerative diseases with no effective treatment. Amyotrophic lateral sclerosis (ALS) is the most common form motor neuron disease (MND), a collective term for a group of neurological disorders characterised by degeneration and loss of motor neurons. ALS is characterised by selective degeneration of the upper and lower motor neurons, leading to muscle wasting and premature death usually due to respiratory failure and paralysis. The median survival for ALS is less than 3 years from diagnosis, but a range of factors can impact on disease duration. The incidence of ALS is approximately 2 per 100,000 people per year. Around 90% of ALS cases are classified as sporadic, with approximately 10% showing a genetic component and familial inheritance. FTD is the second most-common form of early-onset dementia characterised by a progressive loss of neuronal cells in frontal and temporal lobe leading to alterations in cognitive function and personality, leaving patients unable to care for themselves and resulting in death between 2-15 years from disease onset. There are approximately four new cases per 100,000 people, per year. ALS and FTD show a substantial clinical, pathological and genetic overlap, with 40-50% of FTD patients developing some degree of motor dysfunction and approximately 25% of ALS cases clinically diagnosed with FTD. Thus, ALS and FTD are proposed to constitute one disease spectrum with related pathogenic mechanisms. Neuroprotective treatment options in ALS and FTD are extremely limited. Currently, the only licensed drug to treat ALS is the anti-glutamatergic agent riluzole, which prolongs survival in ALS patients by only approximately 3-6 months. There is therefore a need for improved therapeutic intervention in these related neurological diseases.

The most common genetic cause of ALS and FTD is a hexanucleotide repeat expansion of GGGGCC, herein referred to as G4C2 (SEQ ID NO: 5), in the first intron of the chromosome 9 open reading frame 72 (C9orf72) gene, termed C9ALS/FTD. C9ALS/FTD shows autosomal dominant inheritance and incomplete penetrance, with pathogenic repeats ranging from 30 into the thousands. The C9orf72 repeat expansion accounts for up to 40% of familial ALS and 25% of familial FTD, although this can vary between populations. The C9orf72 expansion also accounts for a proportion of sporadic ALS and FTD cases, and has been reported in other neurodegenerative diseases including primary lateral sclerosis, progressive muscular atrophy, corticobasal syndrome, Alzheimer's disease, Parkinson's disease, and Dementia with Lewy Bodies. There are 3 recognised pathogenic mechanisms associated with the C9orf72 repeat expansion: 1) RNA toxicity of the repeat expansion 2) protein toxicity from aberrant repeat associated non-ATG (RAN) translated dipeptide repeat (DPR) protein accumulation, and 3) haplo-insufficiency of the C9orf72 gene. Antisense oligonucleotide therapies targeting C9ORF72 are in clinical trials, and are aimed at reducing the expression of the repeat expansion, thus reducing RNA and DPR toxicity, without affecting the normal expression of C9orf72. How the elevated levels of DNA damage as a consequence of defective DNA repair have been proposed as one method of C9orf72 related RNA and DPR protein mediated cellular toxicity ¹. Thus, genome instability is considered a contributing factor in C9ALS/FTD and mechanisms to resolve this could prove to be beneficial.

RuvBL1 and RuvBL2 (also known as RVB1/RVB2, Pontin/Reptin and TIP49/TIP48) are members of the AAA+ (ATPase associated with diverse cellular activities) family of ATPases. RuvBL1 and RuvBL2 are structurally similar, sharing motifs and domain structure characteristic of the AAA+ superfamily ²⁻⁴. Structural analysis via X-ray crystallography and electron microscopy indicates RuvBL1 and RuvBL2 monomers oligomerize into hetero and homo hexameric rings, which can further stack into a double ring structure ²⁻⁵. The organisation of these oligomeric-hexamers, whether hetero or homo, is likely associated with specific functions of the RuvBL1/2 containing complex, and are also structurally important for the intrinsic ATPase activity of both RuvBL1 and RuvBL2, which hydrolyse ATP via their conserved Walker A and B motifs ⁵⁻⁶. RuvBL1 and RuvBL2 are highly conserved from yeast to mammals, and are paralogous to the bacterial RuvB protein, indicating a role in fundamental cellular processes. Indeed, RuvBL1/2 are components of multiple intracellular protein complexes, and are involved in a range essential cellular pathways including transcriptional regulation, telomerase biogenesis, mitotic assembly, and ribonucleoprotein complex biogenesis (as reviewed in ⁷⁻⁹).

The present disclosure relates to transcription cassettes comprising nucleic acids encoding RuvBL1 and/or RuvBL2 and the use of said vectors in gene therapy for the treatment of Motor Neuron Diseases (MND) such as ALS and other neurodegenerative diseases such as FTD, that result from polymorphic repeat expansions of the GGGGCC (SEQ ID NO: 5) hexanucleotide-repeat sequence in the first intron of the C9ORF72 gene. Expression of said genes which increases RuvBL1 and 2 protein levels is specifically targeted to neuronal cells with RuvBL1 and/or RuvBL2 depleted levels to ameliorate disease.

### STATEMENT OF THE INVENTION

According to an aspect of the invention there is provided an isolated nucleic acid molecule comprising: a transcription cassette comprising a promoter adapted for expression in a mammalian neurone said cassette further comprising a nucleotide sequence encoding an ATPase selected from the group consisting of:
i) a nucleotide sequence as set forth in SEQ ID NO:1 and/ or SEQ ID NO: 2;
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 1 and/or SEQ ID NO: 2 wherein said nucleic acid molecule encodes an ATPase;
iv) a nucleotide sequence that encodes a polypeptide comprising an amino acid sequence as represented in SEQ ID NO: 3 and/or 4;
v) a nucleotide sequence that encodes a polypeptide comprising an amino acid sequence wherein said amino acid sequence is modified by addition deletion or substitution of at least one amino acid residue as represented in iv) above and which has ATPase activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘis the temperature at which 50% of a given strand of a nucleic acid molecule is hybridized to its complementary strand. The following is an exemplary set of hybridization conditions and is not limiting:

Very High Stringency (allows sequences that share at least 90%, 91%, 92%, 93%, 94%, 95%, 96%, 97%, 98% or 99% identity to hybridize)
Hybridization: 5× SSC at 65° C. for 16 hours
Wash twice: 2× SSC at room temperature (RT) for 15 minutes each
Wash twice: 0.5× SSC at 65° C. for 20 minutes each

High Stringency (allows sequences that share at least 80%, 81%, 82%, 83%, 84%, 85%, 86%, 87%, 88%, or 89% identity to hybridize)
Hybridization: 5×-6× SSC at 65° C-70° C. for 16-20 hours
Wash twice: 2× SSC at RT for 5-20 minutes each
Wash twice: 1× SSC at 55° C-70° C. for 30 minutes each

Low Stringency (allows sequences that share at least 50%, 55%, 60%, 65%, 70% or 75% identity to hybridize)
Hybridization: 6× SSC at RT to 55° C. for 16-20 hours
Wash at least twice: 2×-3× SSC at RT to 55° C. for 20-30 minutes each.

In a preferred embodiment of the invention said cassette is adapted for expression in a neurone. Preferably, said neurone is a motor neurone.

In a preferred embodiment of the invention said nucleic acid molecule comprises or consists of a nucleotide sequence as represented in SEQ ID NO: 1 and/or 2.

In a preferred embodiment of the invention there is provided a nucleotide sequence that encodes a polypeptide, or polymorphic sequence variant thereof, comprising an amino acid sequence as represented in SEQ ID NO: 3 and/or 4.

A polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the polypeptides have at least 70% identity, even more preferably at least 75% identity, still more preferably at least 80%, 85%, 90%, 95% identity, and at least 99% identity with most or the full-length amino acid sequence illustrated herein.

In a preferred embodiment of the invention said promoter is a constitutive promoter.

In an alternative embodiment of the invention said promoter is a regulated promoter, for example an inducible or cell specific promoter.

In a preferred embodiment of the invention said promotor is selected from the group consisting of: chicken beta actin (CBA) promoter, chicken beta actin hybrid promoter (CBh), CAG promoter, eF-1a promoter, neuronal and glia specific promoters including, synapsin 1, Hb9, Camkll, MeCP2, and GFAP promoter sequences.

In a further preferred embodiment of the invention said promoter is selected from the group consisting of: MeP229, MeCP2 and JeT promoter sequences.

In a preferred embodiment of the invention said JeT promoter nucleotide sequence comprises or consist of a sequence set forth in

SEQ ID NO 8
GGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGG
ACGCGCCGGGTGTGGCACAGCTAGTTCCGTCGCAGCCGGGATTTGGGTCG
CGGTTCTTGTTTGT.

JeT promoter sequences are known in the art and disclosed in US patent application US2002/0098547, the entirely content of which is hereby incorporated by reference.

According to a further aspect of the invention there is provided an expression vector comprising a transcription cassette according to the invention.

Viruses are commonly used as vectors for the delivery of exogenous genes. Commonly employed vectors include recombinantly modified enveloped or non-enveloped DNA and RNA viruses, for example baculoviridiae, parvoviridiae, picornoviridiae, herpesveridiae, poxviridae, adenoviridiae, picornnaviridiae or retroviridae e.g. lentivirus. Chimeric vectors may also be employed which exploit advantageous elements of each of the parent vector properties (See e.g., Feng, et al (1997) Nature Biotechnology 15:866-870). Such viral vectors may be wild-type or may be modified by recombinant DNA techniques to be replication deficient, conditionally replicating or replication competent. Conditionally replicating viral vectors are used to achieve selective expression in particular cell types while avoiding untoward broad-spectrum infection. Examples of conditionally replicating vectors are described in Pennisi, E. (1996) Science 274:342-343; Russell, and S. J. (1994) Eur. J. of Cancer 30A(8):1165-1171.

Preferred vectors are derived from the adenoviral, adeno-associated viral or retroviral genomes.

In a preferred embodiment of the invention said expression vector is a viral based expression vector.

In a preferred embodiment of the invention said viral based vector is an adeno-associated virus [AAV].

In a preferred embodiment said viral based vector is selected from the group consisting of: AAV2, AAV3, AAV6, AAV13; AAV1, AAV4, AAVS, AAV6 and AAV9.

In a preferred embodiment of the invention said viral based vector is AAV9.

In an alternative preferred embodiment of the invention said viral based vector is a lentiviral vector.

According to a further aspect of the invention there is provided a pharmaceutical composition comprising an expression vector according to the invention and an excipient or carrier.

The expression vector compositions of the present invention are administered in pharmaceutically acceptable preparations. Such preparations may routinely contain pharmaceutically acceptable concentrations of salt, buffering agents, preservatives, compatible carriers and supplementary therapeutic agents. The expression vector compositions of the invention can be administered by any conventional route, including injection or by gradual infusion over time.

The expression vector compositions of the invention are administered in effective amounts. An “effective amount” is that amount of the expression vector that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The expression vector compositions used in the foregoing methods preferably are sterile and contain an effective amount of expression vector according to the invention for producing the desired response in a unit of weight or volume suitable for administration to a patient. The doses of vector administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. Other protocols for the administration of vector compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the expression vector compositions of the invention are applied in pharmaceutically acceptable amounts and in pharmaceutically acceptable compositions. The term “pharmaceutically acceptable” means a non-toxic material that does not interfere with the effectiveness of the biological activity of the active agent. Such preparations may routinely contain salts, buffering agents, preservatives, compatible carriers, and optionally other therapeutic agents' (e.g. those typically used in the treatment of the specific disease indication). When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The pharmaceutical compositions containing the expression vectors according to the invention may contain suitable buffering agents, including acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The expression vector compositions may conveniently be presented in unit dosage form and may be prepared by any of the methods well-known in the art of pharmacy. All methods include the step of bringing the active agent into association with a vector which constitutes one or more accessory ingredients. The preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butanediol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided an expression vector according to the invention for use as a medicament.

According to a further aspect of the invention there is provided an expression vector according to the invention for use in the treatment of a neurodegenerative disease.

In a preferred embodiment of the invention said neurodegenerative disease is associated with polymorphic GlyGlyGlyGlyCysCys (G4C2; SEQ ID NO: 5) repeat expansions in the first intron of the C9orf72 gene.

In a preferred embodiment of the invention said neurodegenerative disease is selected from the group consisting of: amyotrophic lateral sclerosis (ALS) and frontotemporal dementia (FTD) motor neurone disease, frontotemporal lobar dementia (FTLD), Huntington's like disorder, primary lateral sclerosis, progressive muscular atrophy, corticobasal syndrome, Alzheimer's disease and Dementia with Lewy Bodies.

In a preferred embodiment of the invention said neurodegenerative disease is amyotrophic lateral sclerosis (ALS).

In a preferred embodiment of the invention said neurodegenerative disease is frontotemporal dementia (FTD).

According to a further aspect of the invention there is provided a cell transfected with an expression vector according to the invention.

In a preferred embodiment of the invention said cell is a neurone.

In a preferred embodiment of the invention said neurone is a motor neurone.

According to a further aspect of the invention there is provided a method to treat or prevent a neurodegenerative disease comprising administering a therapeutically effective amount of an expression vector according to the invention to prevent and/or treat said neurodegenerative disease.

In a preferred method of the invention said neurodegenerative disease is amyotrophic lateral sclerosis (ALS).

In a preferred method of the invention said neurodegenerative disease is frontotemporal dementia (FTD).

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a aspect, embodiment or example of the invention are to be understood to be applicable to any other aspect, embodiment or example described herein unless incompatible therewith.

An embodiment of the invention will now be described by example only and with reference to the following figures:

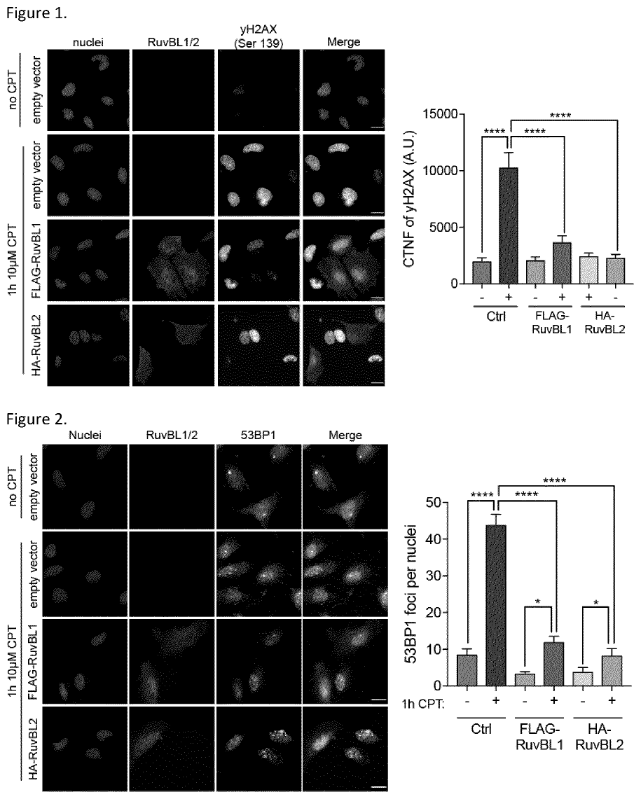

FIG. 1. RuvBL1 and RuvBL2 overexpression reduces nuclear γH2AX accumulation after CPT-induced DNA damage. HeLa cells transfected with empty vector control, FLAG-tagged RuvBL1 or HA-tagged RuvBL2 were treated with 10 μM captothecin (CPT) for 1 h before immunostaining with anti-FLAG or anti-HA and anti-γH2AX (Ser139) antibodies. Levels of nuclear γH2AX are expressed as corrected total nuclear fluorescence (CTNF). Images for no-CPT treated FLAG-RuvBL1 and HA-RuvBL2 overexpressing cells are not shown. (mean±SEM from 2 independent experiments; one-way ANOVA with Tukey's post-test: **** P≤0.0001; Scale bar=20 μm);

FIG. 2. RuvBL1 and RuvBL2 overexpression reduces 53BP1 nuclear foci after CPT-induced DNA damage. HeLa cells transfected with empty vector control, FLAG-tagged RuvBL1 or HA-tagged RuvBL2 were treated with 10 μM captothecin (CPT) for 1 h before immunostaining with anti-FLAG or anti-HA and anti-53BP1 antibodies. Images for no-CPT treated FLAG-RuvBL1 and HA-RuvBL2 overexpressing cells are not shown. The number of 53BP1 nuclear foci were quantified (mean±SEM from 2 independent experiments; one-way ANOVA with Tukey's post-test: *P≤0.05, **** P≤0.0001; Scale bar=20 μm);

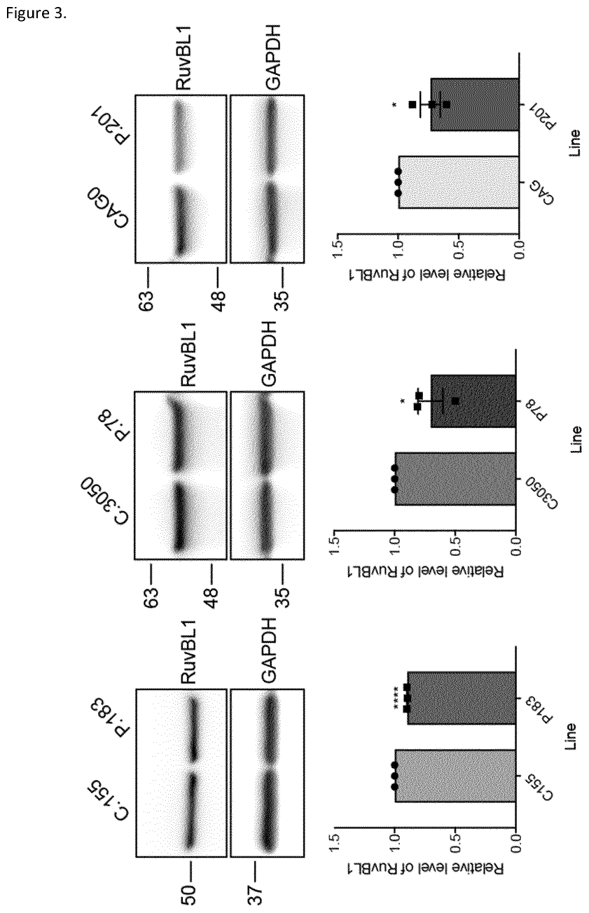

FIG. 3. C9orf72 ALS/FTD patient iNPCs have reduced levels of RuvBL1. RuvBL1 protein levels from 3 C9orf72 ALS/FTD patient iNPC lines (P.183, P.78 and P.201) and their age and sex-matched controls (C.155, C.3050 and C.AGO respectively) were determined by immunoblot. Levels of RuvBL1 were normalized to GAPDH and are shown relative to the age and sex matched control (mean±SEM; unpaired t-test: *P≤0.05, **** P≤0.0001; N=3 independent experiments);

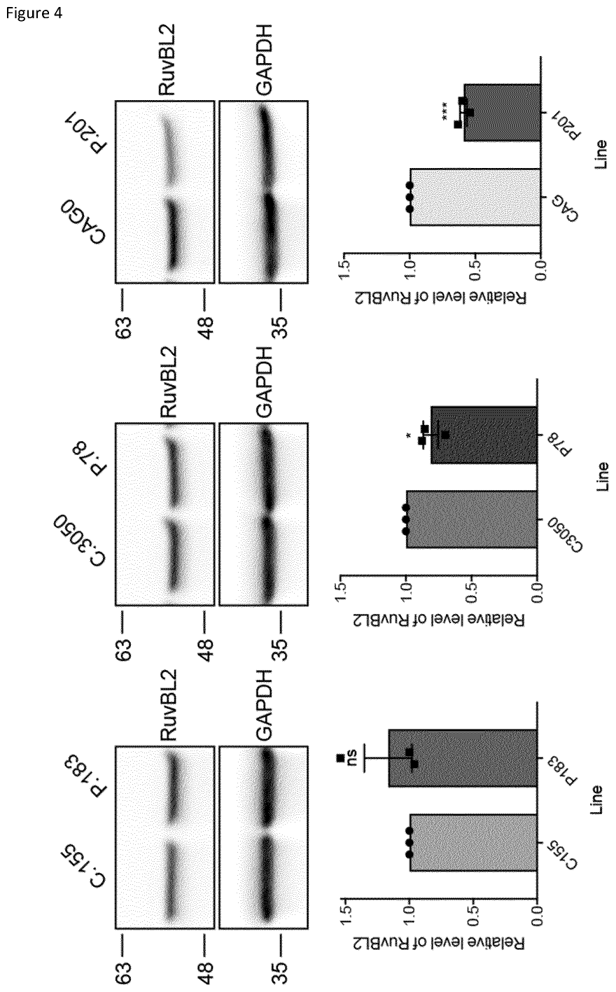

FIG. 4. C9orf72 ALS/FTD patient iNPCs have reduced levels of RuvBL2. RuvBL2 protein levels from 3 C9orf72 ALS/FTD patient iNPC lines (P.183, P.78 and P.201) and their age and sex-matched controls (C.155, C.3050 and C.AGO respectively) were determined by immunoblot. Levels of RuvBL2 were normalized to GAPDH and are shown relative to the age and sex matched control (mean ±SEM; unpaired t-test: ns=non-significant, *P≤0.05, *** P≤0.001; N=3 independent experiments);

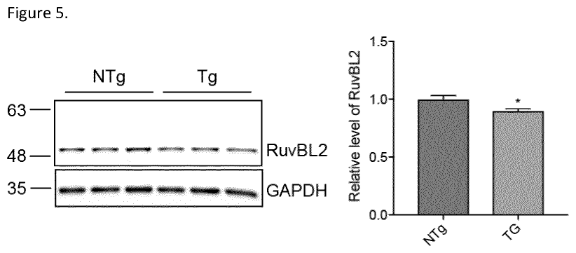

FIG. 5. C9-BAC500 cortical neurons have reduced levels of RuvBL2. Cortical neurons isolated from non-transgenic (NTg) and transgenic (Tg) C9-BAC500 E16 mice embryos were lysed after 10 DIV (days in vitro). RuvBL2 protein levels were determined by immunoblot. Levels of RuvBL2 were normalised to GAPDH and are shown relative to the non-transgenic controls (mean±SEM; unpaired t-test: *P≤0.05; N=3 embryos); and

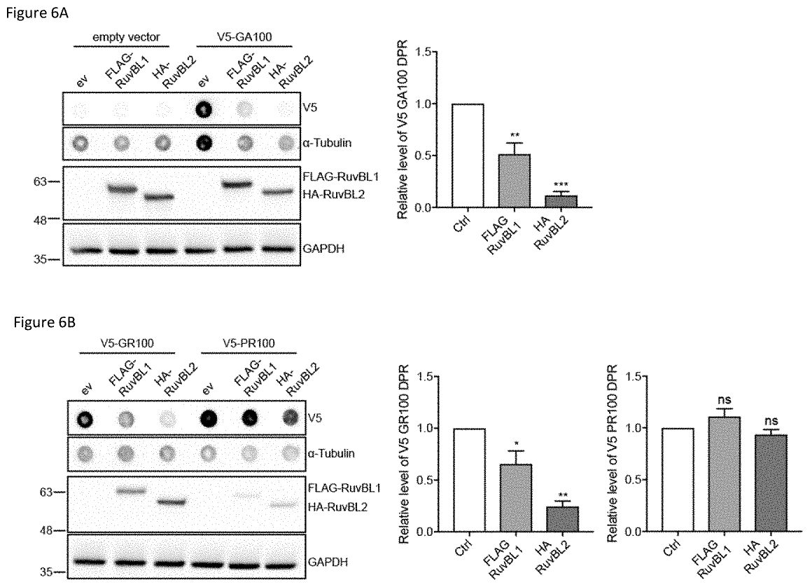

FIGS. 6A-6B. RuvBL1 and RuvBL2 overexpression reduces C9orf72 associated DPR proteins. HeLa cells transfected with (A) empty vector control (ev), V5-tagged GA100, (B) V5-tagged GR100 or V5-tagged PR100 dipeptide repeat expressing plasmids were co-transfected with ev, FLAG-tagged RuvBL1 or HA-tagged RuvBL2. 48 h post transfection cells were lysed and the levels of V5-tagged DPRs determined by dot-blot analysis. Immunoblots blots were also performed to confirm FLAG-RuvBL1 and HA-RuvBL2 overexpression using anti-FLAG and anti-HA antibodies. Levels of V5-tagged DPRs were normalised to α-tubulin and expressed relative to the empty vector control (mean±SEM; unpaired t-test: *P≤0.05, ** P≤0.005, *** P≤0.001; N=3 independent experiments)

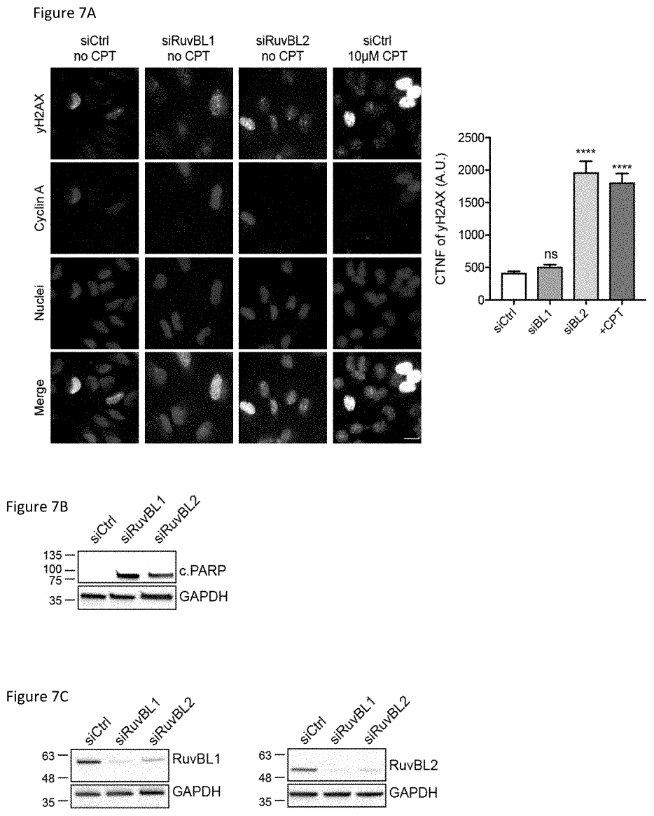

FIGS. 7A-7C. Loss of RuvBL2 leads to DNA damage. HeLa cells were treated with non-targeting control (siCtrl), RuvBL1 (siRuvBL1) or RuvBL2 (siRuvBL2) siRNA and immunostained with anti-γH2AX (Ser139) and anti-Cyclin A antibodies. (A). Cyclin A staining identified cells in G₂ phase of the cell cycle and due to undergo mitosis. Cyclin A positive cells were excluded from the analysis. Levels of nuclear γH2AX in Cyclin A negative cells are expressed as corrected total nuclear fluorescence (CTNF). Treatment of siCtrl cells with 10 μM CPT for 1 h before immunostaining acted as a positive control for increased DNA damage. RuvBL1 and RuvBL2 knockdown increased cleaved PARP-1 (c.PARP) accumulation. (B). RuvBL1 and RuvBL2 knockdown was confirmed by immunoblot. (C). (mean±SEM from 2 independent experiments; one-way ANOVA with Tukey's post-test: **** P≤0.0001; Scale bar=20 μm).

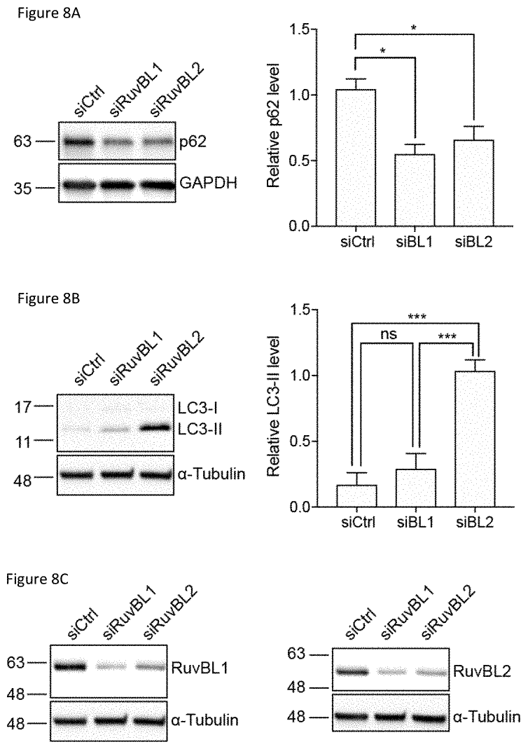

FIGS. 8A-8C. Loss of RuvBL1 and RuvBL2 perturbs basal autophagy. HeLa cells were treated with non-targeting control (siCtrl), RuvBL1 (siRuvBL1) or RuvBL2 (siRuvBL2) siRNA. 4 days post treatment levels of p62 (A) LC3-II (B) were determined by immunoblot. Levels of p62 and LC3-II were normalised against α-Tubulin and are shown relative to the average of the siCtrl samples. RuvBL1 and RuvBL2 knockdown was assessed by western blot (C) (mean±SEM; one-way ANOVA with Tukey's post-test: * P≤0.05, *** P≤0.001; N=4 experiments); and

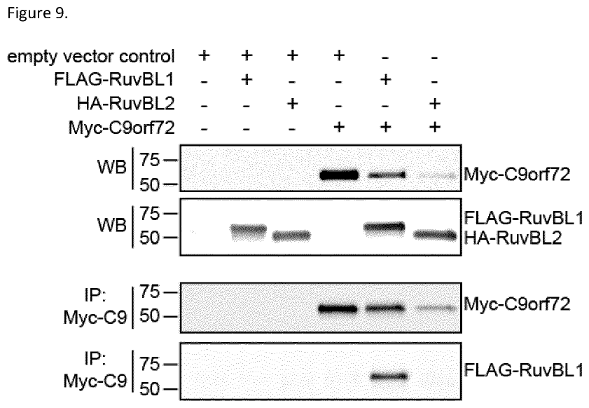

FIG. 9. RuvBL1 interact with C9orf72. Cell lysates of HeLa cells co-transfected with Myc-C9orf72 and either empty vector, FLAG-RuvBL1 or HA-RuvBL2 were subjected to immunoprecipitation with anti-Myc antibodies. Immune pellets (IP: Myc-C9) were probed for Myc-C9orf72, FLAG-RuvBL1 and HA-RuvBL2 on immunoblots.

### SEQUENCE LISTING

The Sequence Listing is submitted as an ASCII text filed in the form of the file name “Sequence.txt” (˜16 kb), which was created on Jul. 6, 2022, and which is incorporated by reference herein.

### Materials and Methods

### Plasmids

pCi-Neo empty vector plasmid was purchased from (Promega), pCMV3 FLAG-tagged RuvBL1 and HA-tagged RuvBL2 were purchased from SinoBiologicals.

Synthetic sequences encoding poly-Gly-Ala, poly-Gyl-Arg and poly-Pro-Arg ×100 DPRs independently of G4C2 repeats were first cloned into pcDNA3.1 using EcoRl/Notl. Synthetic sequences encoding poly-Gly-Ala, poly-Gyl-Arg and poly-Pro-Arg ×100 were subcloned using BamHI/NotI into pCI-neo-V5-N using BcII/NotI. BcII restriction site was previously introduced into pCI-neo-V5-N by site directed mutagenesis using forward ACTCTAGAGGTACCACGTGATCATTCTCGAGGGTGCTATCCAGGC (SEQ ID NO: 6) and reverse GCCTGGATAGCACCCTCGAGAATGATCACGTGGTACCTCTAGAGT (SEQ ID NO: 7) primers.

### Cell Culture and Transfection

HeLa cells were cultured in Dulbecco's modified Eagle's medium (DMEM, SUPPLIER), supplemented with 10% FBS (SUPPLIER) and 100 IU/ml penicillin and 100 IU/ml streptomycin (Sigma) in a 5% CO₂ atmosphere at 37° C. HeLa cells were transfected with plasmid DNA using polyethylenimine (PEI) (stock 1 mM; 3 μl/μg plasmid). Cells were used in experiment 24 or 48 h post DNA transfections. HeLa cells were siRNA transfected using Lipofectamine RNAiMax (Invitrogen) according to the manufacturer's instructions. Cells were used in experiments 4 days after siRNA transfection.

Cortical neurons were isolated from E15 FVB/NJ-Tg(C9orf72)500Lpwr/J (C9 BAC-500, The Jackson Laboratory) embryos and cultured on 6 well tissue culture plates coated with poly-L-lysine in neurobasal medium supplemented with B27 supplement (Invitrogen), 100 IU/ml penicillin, 100 mg/ml streptomycin, and 2 mM L-glutamine. Cells were harvested for immunoblot analysis after 10 days in vitro.

iNPC Production

Induced neural progenitor cells (iNPCs) were derived from human skin fibroblasts as previously described ¹⁰. Human skin fibroblast samples were obtained from Professor Pamela J Shaw from the Sheffield tissue bank. Informed consent was obtained from all subjects before sample collection. Briefly, 10,000 fibroblasts were transduced with lentiviral vectors for OCT3, Sox2, KLF4, and C-MYC for 12 h. Forty-eight hours after transduction, the cells were washed with PBS and fibroblast medium was replaced with NPC medium (DMEM/F-12 with glutamax supplemented with 1% N2, 1% B27, 20 ng/ml FGF-b, 20 ng/ml EGF, and 5 μg/ml heparin. When the cells started changing shape and form neurospheres, they were expanded as neural rosettes. When the iNPC culture was confluent (˜3 weeks), EGF and heparin were withdrawn, and the FGF-b concentration increased to 40 ng/ml. The iNPCs can be maintained for ˜30 passages. iNPCs are not expanded by clone and therefore do not display clonal variability.

### SDS-PAGE and Immunoblotting

Cells were harvested in Trypsin/EDTA (Lonza) and pelleted at 400 xg for 4 min. Pellets were washed once in phosphate buffered saline (PBS). Cell pellets were lysed in ice for 30 min in ice cold RIPA buffer (50 mM Tris-HCl pH 6.8, 150 mM NaCl, 1 mM EDTA, 1 mM EGTA, 0.1% (w/v) SDS, 0.5% (w/v) deoxycholic acid, 1% (w/v) Triton X-100+protease inhibitor cocktail). Lysates were cleared at 17,000 xg for 20 min at 4° C. Protein concentration was measured by Bradford assay (BioRad). Proteins were separated by SDS-PAGE and transferred to nitrocellulose membranes (Whatmann) by electroblotting (BioRad). Membranes were blocked for 1 h in Tris buffered saline (TBS) with 5% fat-free milk (Marvel) and 0.1% Tween-20. Membranes were incubated in primary antibodies in blocking buffer overnight at 4° C. Membranes were washed three times for 10 min in TBS with 0.1% Tween-20 before incubation with secondary antibodies diluted in TBS with 0.1% Tween-20 for 1 h at room temperature.

Membranes were washed again three times for 10 min in TBS with 0.1% Tween-20. Membranes were prepared for chemiluminescent signal detection with Enhanced Chemiluminescent (ECL) substrate according to the manufacturer's instructions. Chemiluminescent signal was detected on a Syngene Gbox and signal intensities were quantified using ImageJ.

### Dot-blotting

For dot blot analysis cells were harvested directly into 2× laemmli loading buffer and diluted 1:2 with distilled H₂O. Lysates were passed 20 times through a 25 G needle to shear genomic DNA before boiling at 95° C. for 3 min. Equal volumes of lysate were loaded to the 96 well Bio-Dot Microfiltration Apparatus (BioRad) and transferred to nitrocellulose membranes under vacuum. Sample wells were washed 3 times in TBS with 0.1% Tween-20 before dismantling. Nitrocellulose membrane was then subjected to clocking and immunoblotting as described above. Chemiluminescent signal was detected on a Syngene Gbox and signal intensities were quantified using ImageJ.

### Antibodies

Primary antibodies used were as follows: rabbit anti-RuvBL1 (Bethyl Laboratories, WB: 1:1,000), rabbit anti-RuvBL2 (Bethyl Laboratories, WB: 1:1,000), rabbit anti-53BP1 (Bethyl Laboratories, IF: 1:500), mouse anti-γH2AX (Merck Millipore, IF: 1:1,000), mouse anti-GAPDH (Merck Millipore, WB: 1:4,000), mouse anti-Tubulin (DM1A, Sigma, WB: 1:10,000), mouse anti-V5 (Invitrogen, WB: 1:5,000), mouse anti-FLAG (M2, Sigma, WB and IF: 1:2,000), mouse anti-HA (HA-7, Sigma, WB and IF: 1:2,000). Secondary antibodies used for immunoblotting were horseradish peroxidase-coupled goat anti-rabbit, and rabbit anti-mouse IgG (Dako; 1:5,000). Secondary antibodies used for immunofluorescence were Alexa fluorophore (488 and 568)-coupled goat/donkey anti-mouse IgG, Alexa fluorophore (488 or 568)-coupled goat/donkey anti-rabbit IgG (Invitrogen; 1:500).

### Immunofluorescence

Cells cultured on glass coverslips were fixed with 3.7% formaldehyde in PBS for 20 min at room temperature or with ice cold methanol:acetone (50:50). Cells were washed twice in PBS before excess formaldehyde was quenched by incubation with 50 mM NH₄Cl in PBS for 20 min at room temperature. Cells were further washed two times in PBS before permeabilising with 0.2% Triton X-100 in PBS for 3 min at room temperature. After washing three times in PBS to remove Triton X-100, cells were blocked in 3% BSA in PBS for 30 min at room temperature before incubation with primary antibodies diluted in 3% BSA-PBS for 1 h at room temperature. After washing three times in PBS, cells were incubated with secondary antibodies diluted in 3% BSA-PBS for 1 h and stained with Hoechst 33342. After a final wash in PBS, cells were mounted to cover slips in fluorescent mounting medium (Dako).

Images were captured using appropriate filtersets (Omega Optical and Chroma Technology) using MicroManager software on a Zeiss Axioplan2 microscope fitted with a Retiga R3 (QImaging) CCD camera, PE-300 LED illumination (CoolLED), and a 63×, 1.4NA Plan Apochromat objective (Zeiss). Illumination intensities, exposure times, and camera settings were kept constant during experiments.

### Image Analysis

Image analysis was performed using ImageJ. 53BP1 puncta were counted in single nuclei using the Particle Analysis facility of ImageJ. Nuclei were defined by the Hochest 33342 stain. Where possible, the cells for analysis were selected based on fluorescence in the other channel. Images were filtered using a Hat filter (7×7 kernel) to extract puncta and thresholded such that the visible puncta within the cell were highlighted, but no background was included. Puncta were counted with the Measure Particles facility of ImageJ. For analysis of γ-H2AX signals, corrected total nuclear fluorescence (CTNF) of the γ-H2AX signal was calculated as CTNF=Integrated Density−(Area of selected nuclei X Mean fluorescence of background readings).

### AAV9 Production

AAV9 viral particles were produced by transfecting human embryonic kidney HEK293T cells and purifying using iodixanol gradient purification method. Briefly, HEK293T cells in thirty T175 flasks were transfected with packaging plasmids pHelper (Stratagene; Stockport, UK), pAAV2/9 (kindly provided by J. Wilson, University of Pennsylvania) and one of the transgene plasmids at 2:1:1 ratio, respectively, using polyethylenimine (1 mg/ml) in serum-free Dulbecco's modified Eagle's medium. At 4 days post-transfection, supernatant containing cell-released virus was harvested, treated with benzonase (10 unit/ml; Sigma, Poole, UK) for 2 hours at 37° C. and concentrated to equal to approximately 24 ml using Amicon Ultra-15 Centrifugal 100 K Filters (Millipore, Watford, UK). Iodixanol gradient containing 15, 25, 40, and 54% iodixanol solution in phosphate-buffered saline (PBS)/1 mmol/l MgCl2/2.5 mmol/l KCl and virus solution was loaded and centrifuged at 69,000 revolutions per minute for 90 minutes at 18° C. After ultracentrifugation, the virus fractions were visualized on a 10% polyacrylamide gel, stained using SYPRO Ruby (Life Technologies, Paisley, UK) according to the manufacturer's guidelines. The highest purity fractions (identified by the presence of the three bands corresponding to VP1, VP2, and VP3) were pooled and concentrated further in the final formulation buffer consisting of PBS supplemented with an additional 35 mmol/l NaCl40 using Amicon Ultra-15 Centrifugal 100 K filters. Viral titers were determined by quantitative PCR assays using primers directed against the transgene and a linearized pAAV-CMV vector as a standard curve.

Transduction of Cortical Neurons with AAV9

To transduce primary cortical neuron cultures, 1.5×10⁵ viral genomes (vg) per cell of AAV9 were added to the culture media after 2-5 days in vitro (DIV). Transduction media was replaced with conditioned media after 4 hours of incubation. Half of the culture media was replaced with fresh media every 3 days. 7-days post-transduction (13 DIV), cells were fixed using 4% paraformaldehyde or methanol:acetone (50:50), or harvested for SDS-PAGE and immunoblot as appropriate.

In-vivo AAV9 Delivery to Mice

All experiments involving mice were conducted according to the Animal (Scientific Procedures) Act 1986, under Project License 40/3739 and approved by the University of Sheffield Ethical Review Sub-Committee, and the UK Animal Procedures Committee (London, UK). The UK Home Office code of practice for the housing and care of animals used in scientific procedures was followed according to Animal (Scientific Procedures) Act 1986. Animals were maintained in a controlled facility in a 12-hour dark/12-hour light cycle, a standardized room temperature of 21° C., with free access to food and water.

For AAV9 delivery into the CSF via cisterna magna, 15 wild-type C57BL/6 mice (n=3 per group) at postnatal day 1 were anesthetized in an induction chamber using 5% isoflurane and oxygen at 3 l/minute before being placed on a red transilluminator (Philips Healthcare “Wee Sight”—product no. 1017920) with their head tilted slightly forward and nose attached to an anesthetic supply. Anesthesia was maintained with 2% isoflurane and oxygen at 0.3 l/minute. A 33-gauge needle attached to a Hamilton syringe and peristaltic pump was lowered approximately 1 mm into the cisterna magna area using stereotaxic apparatus at an angle of 45 degrees, and 1 μl of viral solution (1×1010 vg/μl) was injected at a rate of 1 μl/minute. An equal volume of PBS/35 mmol/l NaCl was used as a control solution.

For tail vein injections of AAV9, animals aged 3-4 weeks old were placed in a warmer environment (31° C.) for up to 15 minutes and then firmly held with the aid of a restraining device. A heat lamp was used to further dilate the lateral veins in the tail, after which mice received a single intravenous dose of 1×10¹² vg per mouse, in a final volume of 100 pL. Non-treated animals were injected with 100 μL of PBS supplemented with 35 mM NaCl.

### EXAMPLE 1

Genome stability is crucial for cell survival and is maintained by the DNA damage response (DDR). Failure of the DDR to rectify damage has been implicated in a range of neurodegenerative diseases^{11,12}. We previously demonstrated that the C9orf72 repeat expansion leads to DNA damage via the formation of RNA/DNA hybrids called R-loops, which in turn lead to DNA double stand breaks (DSBs)¹. Therefore, correcting this genomic instability in C9ALS/FTD is of therapeutic benefit.

RuvBL1/2 containing complexes are involved in a range of cellular processes, including the DDR. As part of the TIP60 and Ino80 complexes, RuvBL1/2 are recruited to DNA damage sites to regulate histone modification, DNA accessibility, DDR signal amplification and, ultimately, repair¹³⁻¹⁷. We therefore first investigated whether elevating RuvBL1/2 levels could promote DNA damage repair. Chemically induced DNA damage in HeLa cells with camptothecin led to nuclear accumulations of the DSB markers yH2AX and 53BP1 (FIGS. 1 and 2). In the presence of both RuvBL1 and RuvBL2 overexpression the level of nuclear yH2AX and the number of 53BP1 foci was significantly reduced, suggesting a more efficient DNA repair response (FIGS. 1 and 2). These data suggest that RuvBL1/2 overexpression could therefore alleviate the elevated DNA damage found in C9ALS/FTD patient neurons

### EXAMPLE 2

If modulating RuvBL1/2 levels in C9ALS/FTD patients was to be considered as a therapeutic approach, we next investigated the endogenous expression of RuvBL1 and RuvBL2 in C9ALS/FTD patient cells. All 3 C9ALS/FTD patients iNPCs showed significantly less RuvBL1 protein compared to their age and sex matched controls (FIG. 3). RuvBL2 protein expression was significantly reduced in 2 out of 3 C9ALS/FTD patients compared to their matched controls (FIG. 4). Similarly, RuvBL2 expression was significantly reduced in the C9 BAC-500 mouse model of C9ALS/FTD (FIG. 5). Thus, these findings strengthen our rationale for increasing RuvBL1/2 expression levels in C9ALS/FTD patients.

### EXAMPLE 3

Recently RuvBL1/2 have been implicated in protein folding and aggregate clearance^{18, 19}. The C9orf72-repeat expansion is aberrantly translated into 5 species of DPR proteins: poly GA, GR, GP, PA and PR. Since these C9orf72 associated DPR proteins form toxic aggregates within cells, we investigated whether RuvBL1/2 overexpression could promote C9ALS/FTD-associated DPR clearance. HeLa cells were co-transfected with either poly GA, GR or PR, considered the three most toxic DPRs, along with empty vector control, FLAG-RuvBL1 or HA-RuvBL2. Overexpression or RuvBL1 and RuvBL2 led to a significant reduction in the amount of GA and GR DPR proteins as quantified by dot blot (FIG. 6A and B). RuvBL1/2 overexpression did not affect PR DPR levels (FIG. 6B). While the precise pathogenic mechanism associated with the C9orf72 repeat expansion is complex, it is increasingly recognised that a combination of RNA toxicity, DNA damage, DPR toxicity and C9orf72 haploinsufficiency may all contribute to the development of disease. These data indicate that RuvBL1/2 overexpression can alleviate the associated DNA damage while simultaneously aiding in the removal of toxic DPR proteins.

### EXAMPLE 4

Previous studies have indicated that reduced levels of RuvBL1/2 can lead to defective DNA damage repair and DNA damage hypersensitivity ¹⁷. Since C9ALS/FTD patient have reduced expression of either RuvBL1 and/or RuvBL2 (FIGS. 3 and 4), we investigated whether loss of RuvBL1/2 would increase DNA damage. HeLa cells were treated with control, RuvBL1 or RuvBL2 targeting siRNA. DNA damage was then measured by quantifying nuclear yH2AX signal. Knockdown of RuvBL1 did not have a significant effect on nuclear yH2AX signal, while knock down of RuvBL2 significantly increase nuclear yH2AX signal similar to that of the CPT-treated positive control (FIG. 7A). Cells were co-stained with cyclin A to discriminate between cells with elevated DNA damage, and cells about to undergo cell division. Although RuvBL1 knockdown did not lead to detectable increases in DNA damage markers, analysis of cleaved PARP-1 protein, a hallmark of apoptotic cell death, indicated that both RuvBL1 and RuvBL2 siRNA were particularly toxic. Since PARP-1 is involved in DNA damage sensing, this cleaved PARP-1 cell death signature was possibly a consequence of elevated and unresolved DNA damage (FIG. 7B). Knockdown of RuvBL1 and RuvBL2 was confirmed by western blot (FIG. 7C).

### EXAMPLE 5

Since RuvBL1 and RuvBL2 are involved in aggregate protein clearance, we next investigated the effect of RuvBL1/2 knockdown on the autophagic degradation pathway. HeLa cells treated with control, RuvBL1 or RuvBL2 targeting siRNA were analysed by western blot for two of the most commonly assessed autophagy associated proteins p62 and LC3-II. P62 is an autophagy receptor protein and delivery autophagy substrates, including protein aggregates, to the autophagosome for lysosomal degradation. Knockdown of both RuvBL1 and RuvBL2 led to a significant reduction in p62 protein levels (FIG. 8A). Further to this, RuvBL1 siRNA led to a small but not significant increase in the amount of LC3-II, while RuvBL2 siRNA significantly increased LC3-II levels (FIG. 8B). RuvBL1 and RuvBL2 knockdown was confirmed by western blot (FIG. 8C). LC3-II protein is directly associated with the autophagosome membrane during autophagy, and is therefore considered a true marker of autophagy induction. These observed differences in p62 and LC3-II after RuvBL1/2 knockdown therefore indicate that loss of RuvBL1 and/or RuvBL2 can perturb normal basal autophagy, potentially disrupting normal protein clearance. Considering that DPR proteins are autophagy substrates ²⁰, a defective autophagy pathway could severely hamper DPR clearance.

Further to this, we have previously demonstrated that the C9orf72 protein is itself involved in autophagy ²¹. This therefore leads to the hypothesis of a toxic feedforward mechanism, whereby haploinsufficiency of C9orf72 leads to defective autophagy, therefore preventing the efficient clearance of the C9orf72-associated DPR autophagy substrates, and leading to their toxic accumulation. The C9orf72 protein is now known to function as part of a complex with SMCR8 and WDR41, and the presence of C9orf72 appears to stabilise SMCR8 as part of this complex²². Indeed, loss of C9orf72 appears to reduce SMCR8 expression and stability ^{23,24. A} wide range of other C9orf72 interacting partners have been described and interestingly a number of mass spectroscopy screens have identified RuvBL1 or RuvBL2 as potential interactors of the C9orf72 complex²⁵⁻²⁷. We therefore investigated whether RuvBL1 and RuvBL2 could interact with C9orf72. HeLa cells were co-transfected with empty vector control or Myc-C9orf72 along with FLAG-RuvBL1 or HA-RuvBL2. Myc-C9orf72 was immunoprecipitated from cell lysates with anti-Myc antibodies, and immune pellets probed for FLAG-RuvBL1 and HA-RuvBL2. An efficient co-immunoprecipitation was observed between C9orf72 and RuvBL1, indicating they are indeed interacting partners (FIG. 9). Taking into account that loss of C9orf72 appears to reduce the stability and expression of its binding partners, this interaction could have implications on the level of RuvBL1 in C9ALS/FTD patients. Again these data support our rationale of increasing RuvBL1/2 levels in patients, given that loss of C9orf72, which is observed in C9ALS/FTD patients, appears to affect binding partner stability.

Together these data support our proposal of increasing RuvBL1/2 levels to alleviate a number of the pathogenic mechanisms associated with the C9orf72 repeat expansion.

### REFERENCES

1 Walker, C. et al. C9orf72 expansion disrupts ATM-mediated chromosomal break repair. Nat Neurosci 20, 1225-1235, doi:10.1038/nn.4604 (2017).
2 Matias, P. M., Gorynia, S., Donner, P. & Carrondo, M. A. Crystal structure of the human AAA+ protein RuvBL1. J Biol Chem 281, 38918-38929, doi:10.1074/jbc.M605625200 (2006).
3 Puri, T., Wendler, P., Sigala, B., Saibil, H. & Tsaneva, I. R. Dodecameric structure and ATPase activity of the human TIP48/TIP49 complex. J Mol Biol 366, 179-192, doi:10.1016/j.jmb.2006.11.030 (2007).
4 Torreira, E. et al. Architecture of the pontin/reptin complex, essential in the assembly of several macromolecular complexes. Structure 16, 1511-1520, doi:10.1016/j.str.2008.08.009 (2008).
5 Lakomek, K., Stoehr, G., Tosi, A., Schmailzl, M. & Hopfner, K. P. Structural basis for dodecameric assembly states and conformational plasticity of the full-length AAA+ATPases Rvb1 . Rvb2. Structure 23, 483-495, doi:10.1016/j.str.2014.12.015 (2015).
6 Gorynia, S. et al. Structural and functional insights into a dodecameric molecular machine—the RuvBL1/RuvBL2 complex. J Struct Biol 176, 279-291, doi:10.1016/j.jsb.2011.09.001 (2011).
7 Huen, J. et al. Rvb1-Rvb2: essential ATP-dependent helicases for critical complexes. Biochem Cell Biol 88, 29-40, doi:10.1139/o09-122 (2010).
8 Nano, N. & Houry, W. A. Chaperone-like activity of the AAA+ proteins Rvb1 and Rvb2 in the assembly of various complexes. Philos Trans R Soc Lond B Biol Sci 368, 20110399, doi:10.1098/rstb.2011.0399 (2013).
9 Jha, S. & Dutta, A. RVB1/RVB2: running rings around molecular biology. Mol Cell 34, 521-533, doi:10.1016/j.molcel.2009.05.016 (2009).
10 Meyer, K. et al. Direct conversion of patient fibroblasts demonstrates non-cell autonomous toxicity of astrocytes to motor neurons in familial and sporadic ALS. Proceedings of the National Academy of Sciences 111, 829-832, doi:10.1073/pnas.1314085111 (2014).
11 McKinnon, P. J. ATM and the molecular pathogenesis of ataxia telangiectasia. Annu Rev Pathol 7, 303-321, doi:10.1146/annurev-pathol-011811-132509 (2012).
12 Obulesu, M. & Rao, D. M. DNA damage and impairment of DNA repair in Alzheimer's disease. Int J Neurosci 120, 397-403, doi:10.3109/00207450903411133 (2010).
13 Jha, S., Gupta, A., Dar, A. & Dutta, A. RVBs are required for assembling a functional TIP60 complex. Mol Cell Biol 33, 1164-1174, doi:10.1128/MCB.01567-12 (2013).
14 Jha, S., Shibata, E. & Dutta, A. Human Rvb1/Tip49 is required for the histone acetyltransferase activity of Tip60/NuA4 and for the downregulation of phosphorylation on H2AX after DNA damage. Mol Cell Biol 28, 2690-2700, doi:10.1128/MCB.01983-07 (2008).
15 Ikura, T. et al. DNA damage-dependent acetylation and ubiquitination of H2AX enhances chromatin dynamics. Mol Cell Biol 27, 7028-7040, doi:10.1128/MCB.00579-07 (2007).
16 Murr, R. et al. Histone acetylation by Trrap-Tip60 modulates loading of repair proteins and repair of DNA double-strand breaks. Nat Cell Biol 8, 91-99, doi:10.1038/ncb1343 (2006).
17 Wu, S. et al. A YY1-IN080 complex regulates genomic stability through homologous recombination-based repair. Nat Struct Mol Biol 14, 1165-1172, doi:10.1038/nsmb1332 (2007).
18 Zaarur, N. et al. RuvbL1 and RuvbL2 enhance aggresome formation and disaggregate amyloid fibrils. EMBO J 34, 2363-2382, doi:10.15252/embj.201591245 (2015).
19 Narayanan, A. et al. A first order phase transition mechanism underlies protein aggregation in mammalian cells. Elife 8, doi:10.7554/eLife.39695 (2019).
20 Boivin, M. et al. Reduced autophagy upon C9ORF72 loss synergizes with dipeptide repeat protein toxicity in G4C2 repeat expansion disorders. EMBO J 39, e100574, doi:10.15252/embj.2018100574 (2020).
21 Webster, C. P. et al. The C9orf72 protein interacts with Rabi a and the ULK1 complex to regulate initiation of autophagy. The EMBO Journal 35, 1656-1627, doi:10.15252/embj.201694401 (2016).
22 Su, M. Y., Fromm, S. A., Zoncu, R. & Hurley, J. H. Structure of the C9orf72 ARF GAP complex that is haploinsufficient in ALS and FTD. Nature 585, 251-255, doi:10.1038/s41586-020-2633-x (2020).
23 Amick, J., Roczniak-Ferguson, A. & Ferguson, S. M. C9orf72 binds SMCR8, localizes to lysosomes and regulates mTORC1 signaling. Mol Biol Cell, doi:10.1091/mbc.E16-01-0003 (2016).
24 Zhang, Y. et al. The C9orf72-interacting protein Smcr8 is a negative regulator of autoimmunity and lysosomal exocytosis. Genes Dev 32, 929-943, doi:10.1101/gad.313932.118 (2018).
25 Sivadasan, R. et al. C9ORF72 interaction with cofilin modulates actin dynamics in motor neurons. Nat Neurosci 19, 1610-1618, doi:10.1038/nn.4407 (2016).
26 Chitiprolu, M. et al. A complex of C9ORF72 and p62 uses arginine methylation to eliminate stress granules by autophagy. Nat Commun 9, 2794, doi:10.1038/s41467-018-05273-7 (2018).
27 Goodier, J. L. et al. C9orf72-associated SMCR8 protein binds in the ubiquitin pathway and with proteins linked with neurological disease. Acta Neuropathol Commun 8, 110, doi:10.1186/s40478-020-00982-x (2020).

1. An isolated nucleic acid molecule comprising: a transcription cassette comprising a promoter adapted for expression in a mammalian neurone, said cassette further comprising a nucleotide sequence encoding an ATPase selected from the group consisting of:
i) a nucleotide sequence as set forth in SEQ ID NO:1 and/or SEQ ID NO: 2;
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 1 and/or SEQ ID NO: 2 wherein said nucleic acid molecule encodes an ATPase;
iv) a nucleotide sequence that encodes a polypeptide comprising an amino acid sequence as represented in SEQ ID NO: 3 and/or 4;
v) a nucleotide sequence that encodes a polypeptide comprising an amino acid sequence wherein said amino acid sequence is modified by addition deletion or substitution of at least one amino acid residue as represented in iv) above and which has ATPase activity.

2. The isolated nucleic acid molecule according to claim 1 wherein said cassette is adapted for expression in a motor neurone.

3. The isolated nucleic acid molecule according to claim 1, wherein said nucleic acid molecule comprises or consists of a nucleotide sequence as represented in SEQ ID NO: 1 and/or 2 or polymorphic sequence variant thereof.

4. The isolated nucleic acid molecule according to claim 1, wherein said nucleotide sequence encodes a polypeptide comprising an amino acid sequence as represented in SEQ ID NO: 3 and/or 4, or polymorphic sequence variant thereof.

5. The isolated nucleic acid molecule according to claim 1, wherein said promoter is a constitutive promoter.

6. The isolated nucleic acid molecule according to claim 1, wherein said promoter is a regulated promoter, for example an inducible or cell specific promoter.

7. The isolated nucleic acid molecule according to a claim 1, wherein said promoter is selected from the group consisting of: chicken beta actin (CBA) promoter, chicken beta actin hybrid promoter (CBh), CAG promoter, eF-1a promoter, neuronal and glia specific promoters including, synapsin 1, Hb9, CamkII, MeCP2, and GFAP promoter nucleotide sequences.

8. The isolated nucleic acid molecule according to claim 1, wherein said promoter is selected from the group consisting of: MeP229, MeCP2 and JeT promoter nucleotide sequences.

9. An expression vector comprising a transcription cassette according to claim 1.

10. The expression vector according to claim 9 wherein said expression vector is a viral based expression vector.

11. The expression vector according to claim 10 wherein said viral based vector is an adeno-associated virus [AAV].

12. The expression vector according to claim 11 wherein said viral based vector is AAV9.

13. The expression vector according to claim 10 wherein said viral based vector is a lentiviral vector.

14. A cell transfected with an expression vector according to claim 9.

15. The cell according to claim 14 wherein said cell is a neurone.

16. The cell according to claim 15 wherein said neurone is a motor neurone.

17. A pharmaceutical composition comprising the expression vector according to claim 9 and an excipient or carrier.

18. The expression vector according to claim 9 for use as a medicament.

19. The expression vector according to claim 9 for use in the treatment of a neurodegenerative disease.

20. The expression vector according to claim 19, wherein said neurodegenerative disease is associated with polymorphic GlyGlyGlyGlyCysCys (G4C2; SEQ ID NO: 5) repeat expansions in the first intron of the C9orf72 gene.

21. The expression vector according to the use of claim 19, wherein said neurodegenerative disease is selected from the group consisting of: amyotrophic lateral sclerosis (ALS) and frontotemporal dementia (FTD) motor neurone disease, frontotemporal lobar dementia (FTLD), Huntington's like disorder, primary lateral sclerosis, progressive muscular atrophy, corticobasal syndrome, Alzheimer's disease and Dementia with Lewy Bodies.

22. The expression vector according to claim 21 wherein said neurodegenerative disease is amyotrophic lateral sclerosis (ALS).

23. The expression vector according to claim 21 wherein said neurodegenerative disease is frontotemporal dementia (FTD).

24. A method to treat or prevent a neurodegenerative disease comprising administering a therapeutically effective amount of the expression vector according to claim 9 to a subject to prevent and/or treat said neurodegenerative disease in the subject.

25. The method according to claim 24 wherein said neurodegenerative disease is amyotrophic lateral sclerosis (ALS).

26. The method according to claim 24 wherein said neurodegenerative disease is frontotemporal dementia (FTD).


SEQUENCE LISTING (8 sequences)

SEQ ID NO: 1 - DNA, 1371 bp, Homo Sapiens
atgaagattgaggaggtgaagagcactacgaagacgcagcgcatcgcctcccacagccacgtgaaagggctggggctggacgagagcggcttggccaagcaggcggcctcagggcttgtgggccaggagaacgcgcgagaggcatgtggcgtcatagtagaattaatcaaaagcaagaaaatggctggaagagctgtcttgttggcaggacctcctggaactggcaagacagctctggctctggctattgctcaggagctgggtagtaaggtccccttctgcccaatggtggggagtgaagtttactcaactgagatcaagaagacagaggtgctgatggagaacttccgcagggccattgggctgcgaataaaggagaccaaggaagtttatgaaggtgaagtcacagagctaactccgtgtgagacagagaatcccatgggaggatatggcaaaaccattagccatgtgatcataggactcaaaacagccaaaggaaccaaacagttgaaactggaccccagcatttttgaaagtttgcagaaagagcgagtagaagctggagatgtgatttacattgaagccaacagtggggccgtgaagaggcagggcaggtgtgatacctatgccacagaattcgaccttgaagctgaagagtatgtccccttgccaaaaggggatgtgcacaaaaagaaagaaatcatccaagatgtgaccttgcatgacttggatgtggctaatgcgcggccccaggggggacaagatatcctgtccatgatgggccagctaatgaagccaaagaagacagaaatcacagacaaacttcgaggggagattaataaggtggtgaacaagtacatcgaccagggcattgctgagctggtcccgggtgtgctgtttgttgatgaggtccacatgctggacattgagtgcttcacctacctgcaccgcgccctggagtcttctatcgctcccatcgtcatctttgcatccaaccgaggcaactgtgtcatcagaggcactgaggacatcacatcccctcacggcatccctcttgaccttctggaccgagtgatgataatccggaccatgctgtatactccacaggaaatgaaacagatcattaaaatccgtgcccagacggaaggaatcaacatcagtgaggaggcactgaaccacctgggggagattggcaccaagaccacactgaggtactcagtgcagctgctgaccccggccaacttgcttgctaaaatcaacgggaaggacagcattgagaaagagcatgtcgaagagatcagtgaacttttctatgatgccaagtcctccgccaaaatcctggctgaccagcaggataagtacatgaagtaa

SEQ ID NO: 2 - DNA, 1392 bp, Homo Sapiens
atggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctaa

SEQ ID NO: 3 - protein, 456 aa, Homo Sapiens
MKIEEVKSTTKTQRIASHSHVKGLGLDESGLAKQAASGLVGQENAREACGVIVELIKSKKMAGRAVLLAGPPGTGKTALALAIAQELGSKVPFCPMVGSEVYSTEIKKTEVLMENFRRAIGLRIKETKEVYEGEVTELTPCETENPMGGYGKTISHVIIGLKTAKGTKQLKLDPSIFESLQKERVEAGDVIYIEANSGAVKRQGRCDTYATEFDLEAEEYVPLPKGDVHKKKEIIQDVTLHDLDVANARPQGGQDILSMMGQLMKPKKTEITDKLRGEINKVVNKYIDQGIAELVPGVLFVDEVHMLDIECFTYLHRALESSIAPIVIFASNRGNCVIRGTEDITSPHGIPLDLLDRVMIIRTMLYTPQEMKQIIKIRAQTEGINISEEALNHLGEIGTKTTLRYSVQLLTPANLLAKINGKDSIEKEHVEEISELFYDAKSSAKILADQQDKYMK

SEQ ID NO: 4 - protein, 463 aa, Homo Sapiens
MATVTATTKVPEIRDVTRIERIGAHSHIRGLGLDDALEPRQASQGMVGQLAARRAAGVVLEMIREGKIAGRAVLIAGQPGTGKTAIAMGMAQALGPDTPFTAIAGSEIFSLEMSKTEALTQAFRRSIGVRIKEETEIIEGEVVEIQIDRPATGTGSKVGKLTLKTTEMETIYDLGTKMIESLTKDKVQAGDVITIDKATGKISKLGRSFTRARDYDAMGSQTKFVQCPDGELQKRKEVVHTVSLHEIDVINSRTQGFLALFSGDTGEIKSEVREQINAKVAEWREEGKAEIIPGVLFIDEVHMLDIESFSFLNRALESDMAPVLIMATNRGITRIRGTSYQSPHGIPIDLLDRLLIVSTTPYSEKDTKQILRIRCEEEDVEMSEDAYTVLTRIGLETSLRYAIQLITAASLVCRKRKGTEVQVDDIKRVYSLFLDESRSTQYMKEYQDAFLFNELKGETMDTS

SEQ ID NO: 5 - protein, 6 aa, artificial sequence
  repeat expansion
GGGGCC

SEQ ID NO: 6 - DNA, 45 bp, artificial sequence
  primer
actctagaggtaccacgtgatcattctcgagggtgctatccaggc

SEQ ID NO: 7 - DNA, 45 bp, artificial sequence
  primer
gcctggatagcaccctcgagaatgatcacgtggtacctctagagt

SEQ ID NO: 8 - DNA, 164 bp, artificial sequence
  promoter sequence
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt